(19) United States
(12) Patent Application Publication
(10) Pub. No.: US 2007/0066532 A1
(43) Pub. Date: Mar. 22, 2007

(54) Compositions and methods for treatment of muscle pain and muscle wasting

(76) Inventor: Hillary D. White, S. Pomfret, VT (US)

Correspondence Address: LICATA & TYRRELL P.C., 66 E. MAIN STREET, MARLTON, NJ, 08053, US

(21) Appl. No.: 11/555,882

(22) Filed: Nov. 2, 2006

Related U.S. Application Data
(63) Continuation of application No. 10/677,673, filed on Oct. 2, 2003.
Application No. 10/677,673 is a continuation-in-part of application No. 10/464,310, filed on Jun. 18, 2003, now abandoned.

(51) Int. Cl. A61K 38/30; A61K 38/27; A61K 31/57

(52) U.S. Cl. 514/12; 514/171

(57) ABSTRACT

Compositions and methods for alleviating the symptoms associated with muscle pain in general or muscle wasting are provided. The compositions are based on use of androgens, either alone or in combination with other hormones, including growth hormone.

### INTRODUCTION

This application is a continuation of U.S. Ser. No. 10/677,673 filed Oct. 2, 2003, which is a continuation-in-part of U.S. Ser. No. 10/464,310 filed Jun. 18, 2003.

### BACKGROUND OF THE INVENTION

The Women's Health Initiative (WHI) clinical trial, whose aim was to prospectively evaluate the risks and benefits of orally administered combination hormone replacement therapy in healthy women using estrogens and medroxyprogesterone acetate, was recently halted (Fletcher, S. W. et al. 2002. J. Amer. Med. Assoc. 288:366-368). The increased risks in coronary heart disease, breast cancer, stroke, and pulmonary embolism outweighed the increased benefits in colorectal cancer, endometrial cancer, hip fractures and death due to other causes, resulting in a small but statistically significant increased risk for the global index of hazard ratios among women taking these hormones. The authors pointed out, however, that their study only evaluated healthy women, not those with symptoms of hormone deficiency. Furthermore, other routes of delivery, e.g. transdermal systems, need to be studied, since it is possible that transdermal delivery may increase benefits and/or decrease risks to these patients. It was noted by the authors of the WHI study that hormone replacement therapy is still considered to be effective for relieving perimenopausal symptoms such as hot flashes.

Most clinical trials evaluating sex hormone replacement therapy have focused on estrogens and progestins, although testosterone replacement therapy in women who may be testosterone deficient is now beginning to be addressed using transdermal delivery systems, e.g. for disease states in which there is stress from chronic disease with loss of muscle mass and chronic fatigue, such as wasting syndrome in women with AIDS (Miller, K. Et al. 1998. J. Clin. Endocrinol. Metab. 83:2717-2725; Javanbakht, M. Et al. 2000. J. Clin. Endocrinol. Metab. 85:2395-2401). Testosterone replacement therapy using transdermal delivery has also been of benefit to men with symptoms of testosterone deficiency, for example in men with Parkinson s disease (Okun, M. S. et al. 2002. Arch. Neurol. 59:1750-1753). There is accumulating evidence that the sex hormones, in particular estrogens, progestins and now testosterone, are important for subjective feelings of well-being and quality of life, parameters that were not assessed in the Women s Health Initiative trial.

U.S. Pat. No. 5,935,949 discloses a method of alleviating the symptoms of fibromyalgia syndrome and chronic fatigue syndrome which involves oral administration of androgens, such as testosterone, to patients. The idea behind the use of testosterone therapy in the treatment of such conditions is that muscle pain and chronic fatigue, primary symptoms in women with fibromyalgia syndrome (FMS), relates, at least in part, to testosterone deficiency, since androgens are known to allow for increased musculature and improvement in fatigue. Indeed, a small decrease in serum free testosterone concentrations has been documented for premenopausal fibromyalgia patients relative to healthy volunteers, but significance was not achieved for postmenopausal women (Dessein, P. H. et al. 1999. Pain 83:313-319). A relationship between testosterone and pain sensation has been previously suggested (Blomqvist, A. 2000. Compar. Neurol. 423:549-551). Accumulating evidence supports the concept that sex hormones can elevate the pain threshold in an individual, for example, during pregnancy (Gintzler, A. R. 1980. Science 210:193-195), when testosterone concentrations, as well as estrogen and progesterone concentrations, are elevated (Bammann, B. L. et al. 1980. Am. J. Obstet. Gynecol. 137:293-298). The theory that testosterone can suppress pain is supported by the discovery of aromatase-positive cells in the spinal cord dorsal horn of higher vertebrates (quail), where initial processing of pain sensation occurs (Evard, H. Et al. 2000. J. Compar. Neurol. 423:552-564). The presence of aromatase, which converts testosterone to 17_-estradiol, is interesting because it is known that estrogen can induce the transcription of opiates in estrogen receptor-positive cells derived from the superficial layers of the spinal dorsal horn (Amandusson, A. et al. 1996. Neurosci. Lett. 196:25-28; Amandusson, A. et al. 1996. Eur. J. Neurosci. 8:2440-2445; Amandusson, A. et al. 1999. Pain 83:243-248), a location that is important for the synthesis of endogenous opiates. Administration of estrogen to ovariectomized female rats has been demonstrated to increase spinal cord enkephalin transcription (Amandusson, A. et al. 1999. Pain 83:243-248), and estrogen receptor-positive cells co-localize with preproenkephalin mRNA (Amandusson, A. et al. 1996. Eur. J. Neurosci. 8:2440-2445). These endogenous opiates act on enkephalinergic neurons to mediate inhibition of nociceptive relay cells, both in primary afferent fibers as well as in pain-modulating fibers descending from the brainstem (Ma, W. Et al. 1997. Neuroscience 77:793-811). Thus, both testosterone and estrogen appear to be important for modulating the sensation of pain. However, the differential importance of androgens versus estrogens in pain sensation relative to gender remains poorly understood.

Testosterone may also act at the level of the brain. Testosterone concentrations were dramatically decreased in the brain and spinal cord of rats in response to pain-inducing subcutaneous injections of formalin into the paw. In these animals, the loss of testosterone in the central nervous system was demonstrated to be due to its metabolism by 5_-reductase to dihydrotestosterone (Amini, H. Et al. 2002. Pharmacol. Biochem. Behav. 74:199-204). These authors pointed out that dihydrotestosterone can be metabolized to 5_-androstane-3_{—},17_-diol, which is an effective modulator of GABA_{A} receptor complexes in the brain. GABA_{A} receptors are found throughout the brain, and actions of GABA_{A} receptor modulators in the limbic system, specifically in the amygdala, are associated with feelings of fear. The GABA_{A} receptor ion channel complex is one of the most important inhibitory ion channels in the brain. Thus, testosterone may be important not only for modulation of pain but also for feelings of emotional well-being via binding of its metabolites to the neurosteroid site of the GABA_{A} receptor, although this remains to be demonstrated.

Other hormones such as growth hormone may also play a role in the pathogenesis and symptoms of fibromyalgia and chronic fatigue. For example, studies have shown that fibromyalgia patients fail to exhibit a proper growth hormone response to acute exercise, a response that is likely related to increased levels of somatostatin a powerful inhibitor of growth hormone synthesis (Crofford, L. J. et al. 2002. Arthr. Rheumat. 46:1136-1138; Paiva, E. S. et al. 2002. Arthr. Rheumat. 46:1344-1350). It is well known that testosterone increases growth hormone secretion. Growth hormone secretion is reduced in senescence beyond the reduced levels of secretion seen in adult life after puberty. This reduction is thought to relate to the decreased lean body mass to adipose mass ratio known to occur in some individuals in senescence. Thus, increased somatostatin levels may reflect decreased anabolism and decreased muscle mass due to decreased testosterone and growth hormone concentrations in fibromyalgia patients. As a result, therapy with growth hormone may improve the condition of patients with fibromyalgia.

### SUMMARY OF THE INVENTION

An object of the present invention is a method of alleviating symptoms of muscle pain comprising administering to a patient suffering from muscle pain an effective amount of a composition comprising an androgen and a growth hormone so that the symptoms are alleviated.

### DESCRIPTION OF THE DRAWINGS

FIG. 1 depicts the levels of total testosterone in blood of the patients, an average of the group, over time on day 1 (shown with circles) and day 28 (shown with squares).

FIG. 2 depicts the results of the tender point evaluations pre-treatment (day 0) and at the end of the study (day 28). The results reported are levels of pain on a scale of 0 (no pain) to 10 (highest level of pain).

FIG. 3 depicts the results of the dolorimetry assessment of tender point pain pre-treatment (day 0) and at the end of the study (day 28).

FIG. 4 depicts the severity of symptoms/conditions associated with fibromyalgia and chronic fatigue on a scale of 1 to 10 (10 being the highest increased level) on day 1 versus day 28 of the study. The symptoms/conditions assessed included libido, muscle pain, tiredness, headache severity, headache Frequency, stiffness, sleeplessness, fatigue upon awakening, anxiety, and depression.

### DETAILED DESCRIPTION OF THE INVENTION

The syndrome of chronic fatigue has received much attention lately. No physical finding or laboratory test can be used to confirm diagnosis of chronic fatigue syndrome. However, this syndrome is generally characterized by fatigue persisting or relapsing for more than six months occurring concurrently with at least four or more of the following symptoms: impaired memory or concentration, sore throat, tender cervical or axillary lymph nodes, muscle pain, multi-joint pain, new headaches, unrefreshing sleep, and post exertion malaise. Early studies suggested an infectious or immune dysregulation mechanism for the pathophysiology of chronic fatigue syndrome. More recent studies have shown that neurologic, affective and cognitive symptoms also frequently occur.

Fibromyalgia (also referred to as fibrositis) is one of the most common rheumatic syndromes in ambulatory general medicine affecting 3-10% of the general population. Most patients with Fibromyalgia Syndrome (FMS) are women, and of these patients, approximately 50-75% are women in their peri-postmenopausal years, aged 40-60. Approximately 2-5% of peri/post menopausal women are affected by FMS, with some estimates ranging from 0.5 to 20%. This disease is characterized by chronic widespread musculoskeletal pain syndrome with multiple tender points, fatigue, headaches, lack of restorative sleep and numbness. Fibromyalgia shares many features with chronic fatigue syndrome including an increased frequency in peri/post menopausal woman, absence of objective findings and absence of diagnostic laboratory tests. Further, these conditions have overlapping clinical features including chronic fatigue, headaches and lack of restorative sleep with musculoskeletal pain predominating in fibromyalgia and apparent increased susceptibility or hyperimmunologic responsiveness to infection predominating in chronic fatigue syndrome.

Various treatments for chronic fatigue syndrome including acyclovir, oral and vaginal nystatin and fluoxetine have been tried with little success. Placebo-controlled trials have demonstrated modest efficacy of amitriptyline, fluoxetine, chlorpromazine, or cyclobenzaprine in treating fibromyalgia. Exercise programs have also been suggested as beneficial in both conditions. Accordingly, there is clearly a need for better treatments for these debilitating conditions. It has now been found that transdermal administration of hormones, including androgens, can alleviate symptoms in patients suffering from FMS or CFS. By “androgen therapy” it is meant to include administration of a single androgen or a combination of androgens. By “alleviate” it is meant to make less hard to bear, reduce or decrease, or lighten or relieve patients of the symptoms of FMS of CFS. By “symptoms” of FMS or CFS it is meant to include muscle pain and atrophy, chronic fatigue, lack of restorative sleep, increased susceptibility to infection and headaches resulting from FMS or CFS.

A clinical trial was performed to investigate the pharmacokinetics and efficacy of transdermal delivery of hormones for treatment of fibromyalgia. Women were recruited by institutional review board-approved advertising. Subjects aged 40-55 and diagnosed for fibromyalgia using American College of Rheumatology criteria (11/18 bilateral tender points above and below the waist, chronic fatigue, etc., (Wolfe, F. et al. 1990. Arthrit. Rheumat. 33:160-172) were selected for the study if they fit additional criteria. Women were included if, in addition to meeting all other criteria, they agreed to keep their medicines unchanged during the study (decreases in analgesics were permitted). Women taking hormone replacement therapy were enrolled if they agreed to come off hormone therapy at least 2 weeks prior to, and for the duration of, the study, in addition to meeting other eligibility criteria. Pre- or peri-menopausal women were required to have adequate alternative contraception, a negative pregnancy test, and treatment was started within the follicular (proliferative) phase of the menstrual cycle. Patients were included if they were willing to exercise 20 minutes a day, 5 days per week during therapy, to promote the effects of testosterone; this was a requirement put in place by the Institutional Review Board.

Children, pregnant women, and women on hormone therapy, hormone contraceptives or infertility drugs were excluded. Women were excluded from the study if they reported undiagnosed vaginal bleeding, had a body mass index BMI >30, admitted to ethanol or illicit drug abuse, had active thrombophlebitis, breast cancer, hypertension (BP>160 systolic/95 diastolic with or without medication, after sitting 5 minutes), or major skin disease, acne or hirsutism. Prior to enrollment, study patient blood was tested for the following general health criteria (exclusion criteria in parentheses): cardiac risk factors by lipid profile—total fasting cholesterol (>240 mg/dL), high density lipoprotein (<35 mg/dL), low density lipoprotein (>210 mg/dL), triglyceride (>300 mg/L); hepatic function by alanine aminotransferase (>1.5×N, normal at 0-40 U/L), alkaline phosphatase (>2×N, normal at 40-120 U/L), aspartate aminotransferase (>1.5×N, normal at 10-30 U/L), serum albumin (>N, normal at 3.2-5.2 g/dL), total bilirubin (>N, normal at 0.2-1.3 mg/dL), and direct (conjugated, soluble) bilirubin (>N, normal at 0.0-0.3 mg/dL); kidney function by blood urea nitrogen (>2×N, normal at 8-18 mg/dL) and serum creatinine (>N, normal at 0.7-1.2 mg/dL) tests; hematological function was assessed by complete blood cell count including testing for hemoglobin (normal, 12-16 g/dL). Blood tests and physical exam at the end of the study were performed to assess whether testosterone therapy adversely affected the general health of the study patient. Serum total testosterone (>0.4 ng/mL) and FSH (<22 IU/L) were tested as well (8 AM after overnight fasting), to confirm patients had concentrations of testosterone in the lower half of the reference range (2 patients out of 18 were excluded based on testosterone concentrations) and to determine their postmenopausal status. FSH concentrations <22 IU/L indicated premenopausal or perimenopausal status and thus the need for adequate contraception, unless the patient had undergone bilateral oophorectomy. Testosterone serum concentrations were tested at 8 AM due to the small circadian rhythm of circulating androgens. The most frequent exclusion criterion was for BMI >30. Patients were required to stop taking St. John s wort, since St. John s wort is known to induce catabolism of hormones by activating CYP3A, a detoxifying enzyme complex in the liver. Twelve patients who fit the eligibility criteria, above, were scheduled for physical exams including tender point assessment, verification of fibromyalgia diagnosis, and assessment of general health.

On day 1, blood was drawn by venipuncture at 0, 1, 2, 3, 4, 6, 8, 10, 12 and 24 hrs for 24 hr pharmacokinetic profiling of baseline testosterone serum concentrations. Testosterone gel, 0.75 g 1% w/w, was applied by the patient to their lower abdominal skin just after the zero time point blood draw (8 AM). The patient also filled out a pain assessment questionnaire form and was given packets of testosterone gel for 8:00 AM daily application to lower abdominal skin, instructions for use and a patient medication log and exercise log for 28 days of therapy. On day 28, the blood draws for 24 hr pharmacokinetic profiling were repeated, and a follow-up exam was repeated at the end of the 28 days of therapy.

The delivery vehicle for this study was a gel formulation. It was chosen for use as a goal of the study was to identify a transdermal delivery system for hormones that would result in effective levels of hormones in blood as a way to reduce side effects of androgen therapy. The gel used for this study was a 1% w/w testosterone gel, USP grade. The daily gel dose applied was 0.75 grams; an expected bioavailability of 10% would deliver 0.75 mg testosterone over 24 hr. The gel was formulated for women by Bentley Pharmaceuticals, Inc. (North Hampton, N.H.) using good manufacturing practice standards, and is colorless, comfortable on the skin, and non-staining.

Testosterone concentrations were determined by enzyme linked immunoassay (EIA, Diagnostic Systems Laboratories or DSL, Inc, Webster, Tex.), where serum testosterone from study subjects competed with enzyme-linked testosterone bound to anti-testosterone mAb. This assay system was designed to detect the lower concentrations of testosterone found in women as well as concentrations in the upper ranges. Free testosterone concentrations were determined by EIA using an anti-testosterone antibody that recognizes the unbound testosterone in the test sample, and has low affinity for sex hormone binding globulin and albumin. For the purposes of determining mean testosterone concentrations, times were based on the nearest hour. Of the 240 time points taken for the pharmacokinetic data (10 time points per individual×2 sets per individual×12 individuals), 1 time point was missed (#012, 4 hr point) and 3 additional time points were in between the standard times for taking blood (#010, 8 hr point; #012, 4 hr and 10 hr points). Values for these time points were derived by interpolation for the purposes of deriving mean testosterone concentrations. A noncompartmental pharmacokinetic analysis using WinNonlin Pro (Pharsight, Mountain View, Calif.) used the exact time points recorded for all the patients.

In order to determine the efficacy of the treatment for reducing symptoms of fibromyalgia, patients filled out questionnaire forms on day 1 and again at the end of therapy on day 28 to assess pain. The patient questionnaire was based on a published and validated Fibromyalgia Impact Questionnaire as well as other accepted criteria for fibromyalgia patient assessment (Wolfe, F. et al. 1990. Arthrit. Rheumat. 33:160-172; Goldenberg, D. Et al. 1996. Arthrit. Rheumat. 39:1852-1859; Burckhardt, C. S. et al. 1991. J. Rheumatol. 18:728-733), and used a 100 mm visual analog scale (VAS). Tender point exams were administered by a qualified rheumatologist experienced in treating women with fibromyalgia, and involved applying approximately 9 pounds of pressure at each tender point and asking whether the patient felt pain. This practice is in accordance with criteria specified by the American College of Rheumatology. Exams were administered just prior to Day 1 of therapy (and therefore designated as pretreatment ), and at the end of therapy. The pretreatment tender point assessment was performed on all patients within 1 week before the start of therapy. Dolorimeter readings were taken from the bilateral second costochondral junction and trapezius tender points, for comparison, in 11 of the 12 study subjects.

Pharmacokinetic analysis of serum testosterone concentration data was carried out using WinNonlin Pro software, using the noncompartmental model with extravascular input. Differences between Day 1 and Day 28 maximum plasma concentrations (Cₘₐₓ) and area under the curve (AUC) of a plot of plasma concentrations over time were assessed by calculating individual subject Day 28 minus Day 1 data and estimating 95% confidence intervals of this difference to determine if significance (p<0.05) was reached. Tender point data evaluations were analyzed by Student s t test (paired, 2-tailed).

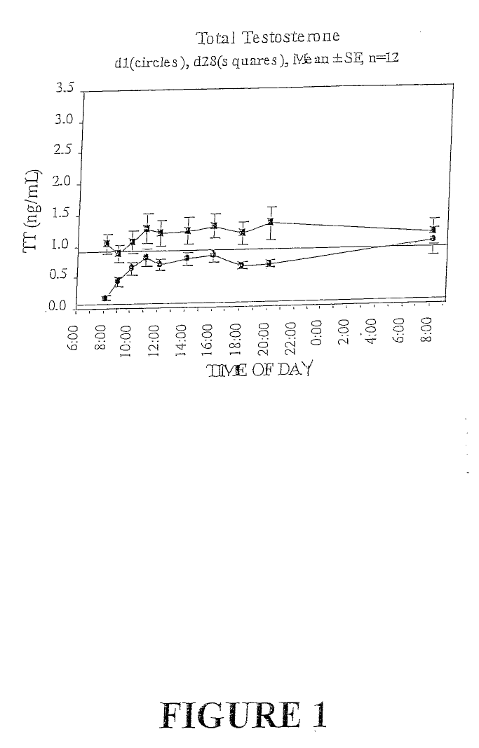

Analysis of the blood testosterone concentration data revealed that serum total testosterone concentrations were reliably increased in fibromyalgia patients in response to testosterone gel hormone replacement therapy. Serum free testosterone concentrations vs time data for Day 1 and Day 28 are shown in FIG. 1. Comparison of the serum testosterone data to standard reference ranges for the concentration of total testosterone in serum from women confirmed that the fibromyalgia patients in this study initially had total testosterone concentrations in the lower half of the reference ranges. However, the mean serum concentration of total testosterone 24 hr after application of the first dose of hormone on Day 1 was significantly higher than the mean serum concentration for time zero on Day 1 (FIG. 1, p=0.01), indicating that serum concentrations were sustained, on average, early on during the 28 day time course. Steady state concentrations were reached by day 28, as evidenced by the similar mean concentrations at the beginning and end of the 24 hr sampling (see FIG. 1). There was variation in the 24 hr profiles for serum testosterone when analyzed on an inter-individual basis, consistent with the complex regulation known for this hormone. Summary pharmacokinetic parameter analysis demonstrated significantly increased mean total testosterone maximum concentration in response to testosterone therapy: Cₘₐₓ was 1.92 ng/mL on day 28 compared with 1.21 ng/mL on day 1, p<0.05. Significantly increased mean total testosterone area under the curve values (assessed over the 24 hr profiling time period) were also found: AUC was 28.75 ng-h/mL on day 28 compared with 18.36 ng-h/mL on day 1, p<0.05. Considered together the pharmacokinetic data demonstrated that with therapy, mean serum total testosterone concentrations initially rose quickly over the first 3 hours and were then reliably sustained over time. In addition, mean serum concentrations were raised from the lower boundary of the reference range to just above the upper end of the reference range for premenopausal women.

Concentrations of free testosterone in serum were also examined and subjected to pharmacokinetic analysis. Results similar to total testosterone results were obtained. However, two of the twelve patients had unusually high concentrations of free testosterone prior to, and throughout, the course of therapy. Individual profiles for the remainder of the patients showed concentrations that increased from the postmenopausal range to the premenopausal and upper postmenopausal reference range. Summary pharmacokinetic parameter analysis showed a mean free testosterone Cₘₐₓ of 4.69 pg/mL on day 28 compared with 3.68 pg/mL on day 1 (p>0.05) and a mean free testosterone AUC of 71.38 pg-h/mL on day 28 compared with 54.35 pg-h/mL on day 1 (p>0.05). Free testosterone Cₘₐₓ and AUC were increased with therapy, as evidenced by subtraction of the day 1 baseline from day 28 values, but statistical significance was not achieved in these pharmacokinetic parameters due to the two individuals with exceptionally high free testosterone concentrations. The high concentrations of free testosterone in those two patients contrasted with the normal total testosterone profiles for these particular individuals, raising the possibility that these high free hormone concentrations may have resulted from low sex hormone binding globulin concentrations in their serum, although other explanations exist. The only medication or supplement reported by both of these study subjects, and not used by any other subjects, was ginger root. (It is not known if ginger root interferes with the enzyme linked immunoassay for free testosterone, or with sex hormone binding globulin metabolic or binding parameters.)

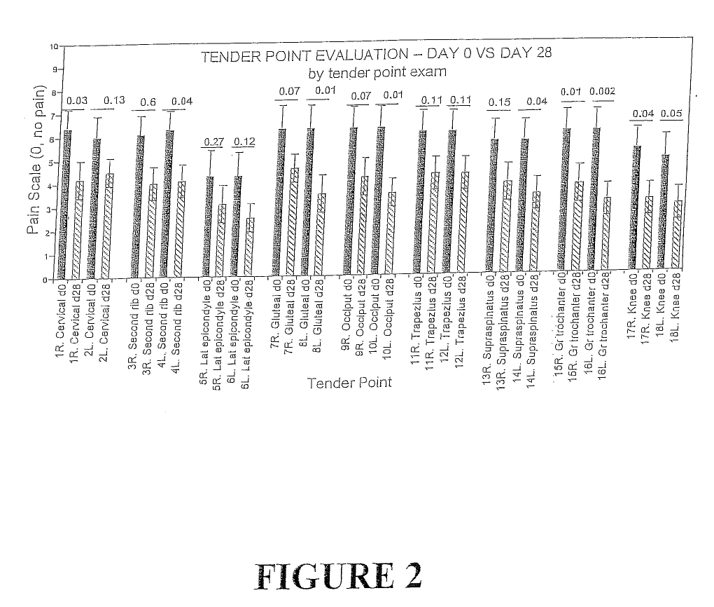

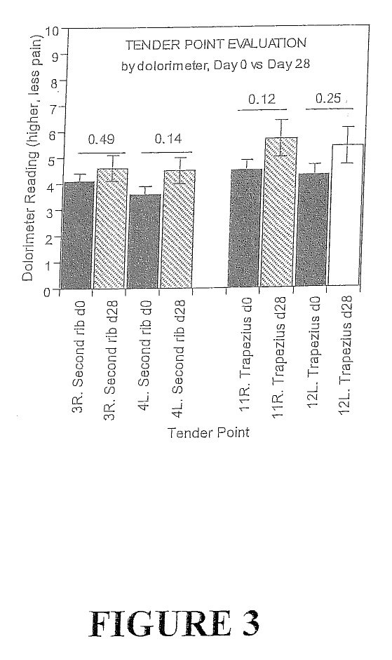

Analysis of the tender point pain data showed that transdermal testosterone gel therapy was associated with decreased subjective assessments of pain. Using a pain scale of 0 to 10, where zero is no pain, there were mean decreases in pain for every tender point, with statistical significance achieved in 9 of 18 categories assessed (categories assessed are listed below in Table 1; results shown in FIG. 2. Using a dolorimeter to assess pain at the same office visit, pain responses were quantitated for the bilateral second costochondral junction and bilateral trapezius tender points (FIG. 3). Individual response values ranged from 2 to 9. Mean dolorimeter values for the pressure at which patients reported pain were higher at the end of 28 days of testosterone treatment, which would be expected if therapy increased thresholds of pain, although the dolorimetry results did not reach statistical significance.

TABLE 1
Tender Points Evaluated

| Tender | Tender | | Lay |
|---|---|---|---|
| Point # | Point | Description | Description |
| 1-2 | lower | bilateral lower cervical | at the base |
| | cervical | (paraspinals) at the | of the neck |
| | | anterior aspect of the | in the back |
| | | intertransverse spaces at | |
| | | C5-7 | |
| 3-4 | second | bilateral at the second | on the |
| | rib | costochondral junction | breast bone |
| | | (rib-cartilage) just | |
| | | lateral to the junction | |
| | | of the upper surface | |
| 5-6 | lateral | bilateral lateral | on the outer |
| | epicondyle | epicondyle in forearm, | edge of the |
| | | 2 cm distal to the | forearm about |
| | | epicondyles | an inch below |
| | | | the elbow |
| 7-8 | gluteal | bilateral gluteal in the | on the outside |
| | | upper outer quadrant of | of the hip |
| | | buttock in the anterior | |
| | | fold of muscle | |
| 9-10 | occiput | bilateral occiput at | At the base |
| | | the insertion of the | of the skull |
| | | suboccipital muscle | beside the |
| | | | spinal column |
| 11-12 | trapezius | bilateral trapezius at | on top of the |
| | | midpoint of the upper | shoulder toward |
| | | border | the back (flat |
| | | | triangular |
| | | | muscle post, |
| | | | neck, shoulder) |
| 13-14 | supraspinatus | bilateral supraspinatus | over the |
| | | at its origin above the | shoulder |
| | | scapular spine near the | blade |
| | | nedial border | |
| 15-16 | greater | bilateral greater | at the top |
| | trochanter | trochanter posterior | of the hip |
| | | to the trochanteric | |
| | | prominence | |
| 17-18 | knee | bilateral knee at the | on the fat |
| | | medial fat pad just | pad over |
| | | proximal to the joint | the knee |
| | | line | |

1-8 anterior, 9-18 posterior

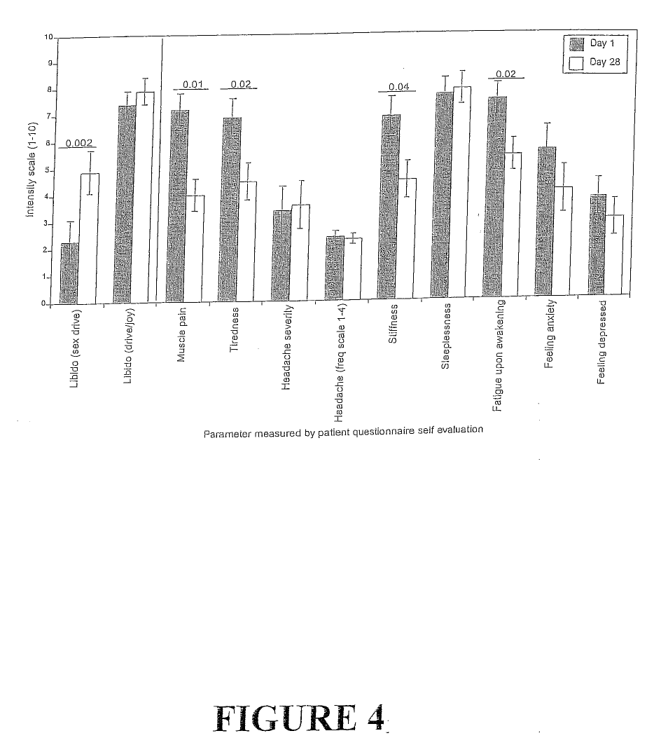

Pain parameters were also evaluated by patient questionnaire using a visual analog scale (VAS) from 0-10 (FIG. 4). Libido (sex drive) was increased in response to testosterone treatment. Muscle pain, tenderness, stiffness and fatigue upon awakening were all decreased during testosterone treatment. These findings are consistent with the idea that restoration of premenopausal serum testosterone concentrations relieves symptoms that most specifically relate to testosterone deficiency, e.g. loss of sexual desire, loss of muscle function and increased fatigue. Blood tests and physical exam at the end of the study verified testosterone therapy did not adversely affect the general health of the study patient, and no study patient reported any adverse events that were attributable to the treatment.

Most trials involving hormone replacement therapy have used derivatives of hormones naturally found in women. These derivatized hormones have been promoted because of their patentability and their extended half life. Androgens are no exception since the androgen hormone most prescribed for women is methyltestosterone, where methylation at the C-17 position increases its oral bioavailability. A subset of patients do not tolerate derivatized hormones very well, however. Non-derivatized exogenous hormones that are structurally identical to endogenous hormones have short plasma/serum half lives that range from 10-100 minutes, making oral administration of native hormones problematic. Investigators have begun to develop transdermal delivery systems, which provide sustained delivery while minimizing hepatotoxicity. A testosterone skin patch has been effective in HIV seropositive women with wasting syndrome (Miller, K. et al. 1998. J. Clin. Endocrinol. Metab. 83:2717-2725; Javanbakht, M. et al. 2000. J. Clin. Endocrinol. Metab. 85:2395-2401), but the skin patch causes topical skin irritation in many women, making its use problematic.

The present invention involves use of a testosterone formulated as a gel in a concentration that is appropriate for women. The data have shown this formulation to provide effective systemic delivery of testosterone in patients with fibromyalgia. 28 days of therapy with 0.75 g 1% (w/w) testosterone gel per day raised serum concentrations of total and free testosterone in fibromyalgia patients to concentrations approximating those in premenopausal women. At this dose, patients showed significantly decreased muscle pain, decreased stiffness, decreased fatigue and increased libido in response to testosterone therapy. Tender point pain was decreased, as well. These results, from both the pharmacokinetic and pain assessment standpoints, support the use of testosterone replacement therapy to treat individuals with fibromyalgia syndrome.

Accordingly, androgen therapy provides a useful means for alleviating symptoms associated with FMS or CFS in women preferably of peri/post menopausal age. By peri/postmenopausal age it is most often meant to be approximately 40 to 60 years of age. Women outside of this range may also benefit since these syndromes have been known to be present in women 20 to 60 years of age. Further, since muscle pain and chronic fatigue are symptoms that can be common to conditions other than fibromyalgia and chronic fatigue syndrome, the present invention is also a treatment for alleviating symptoms of chronic fatigue and muscle pain in patients due to disease in general, accident or aging. The muscle pain in particular may be contemplated to result from atrophy of muscles that is known to occur with aging or long-term confinement to bed, or any condition that limits the amount of activity of a patient and that leads to muscle atrophy or wasting of muscle tissue.

In a preferred embodiment, the androgen administered comprises testosterone, an active metabolite of testosterone such as dihydrotestosterone or androstenedione or a testosterone derivative such as methyltestosterone, testosterone enanthate or testosterone cypionate. Examples of available pharmacologic preparations of androgens believed to be useful in this invention include, but are not limited to danazol, fluoxymesterone, oxandrolone, methyltestosterone, nandrolone decanoate, nandrolone phenpropionate, oxymethalone, stanozolol, methandrostenolone, testolactone, pregnenolone and dehydroepiandrosterone (DHEA).

In the present invention, the androgens are administered transdermally in a gel formulation. This formulation has advantages over current oral methods as well as transdermal patch methods that include improved bioavailability and a low side effect profile. In a preferred embodiment, a combination of androgens such as testosterone or a testosterone derivative and DHEA can be administered to alleviate both the muscular and neurological symptoms of FMS or CFS.

As will be obvious to those of skill in the art upon this disclosure, other pharmaceutically acceptable androgen therapies can be used. Effective amounts and routes by which the androgen or combination of androgens can be administered in the present invention can be routinely determined by those skilled in the art in accordance with other uses for androgen therapies.

The composition of the present invention comprises, in addition to the aforementioned androgen/anabolic agent, co-treatment with a pharmaceutically effective amount of growth hormone elicitor or effector, either growth hormone or an agent that is known to release growth hormone in effective amounts, i.e., a growth hormone releasing agent ( GRF ). GRF is an acronym based on the existence of an endogenous hormone known as GHRH. Other agents include GHrelin or a growth hormone releasing peptide or analog (GHRP; GHRP-6, or hexarelin, His-DTrp-Ala-Trp-DPhe-Lys, and GHRP-2, or Dala-D-2-NaI-Ala-Trp-Dphe-Lys are examples) , which have been shown to release effective amounts of growth hormone. The natural rhythm of growth hormone release from the pituitary gland results in release of insulin-like growth factor (IGF-1), which in general, is considered to be the causal agent that determines the course of hormonal regulation and balance in processes such as adipogenesis and myogenesis. The hormonal effector, then, for the purpose of this invention, is also prophetically considered to be any peptide or peptidomimetic agent that directly acts to release this secondary anabolic growth factor, (IGF-1), not necessarily through the intermediary route of secretion of growth hormone itself. Although the indirect growth hormone route is preferred to elicit IGF-1, the latter route to directly release IGF-1 also is included by example.

In another embodiment of the present invention, the composition comprises a pharmaceutically effective amount of a growth hormone or, more preferably, a growth hormone-releasing agent, or an elicitor of IGF-1 secretion, coupled with androgen treatment and such combined treatment being capable of counteracting the deleterious effects of aging, such as, for example, muscle weakness, body fat increases, and skin fragility in adults. Essentially any suitable growth hormone-releasing agent may be employed in combination with any androgen, preferably one such as testosterone that possesses strong anabolic activity. Other anabolic agents that are not thought of as androgenic agents, or do not possess maximal androgenic activity may be used, as long as they have appreciable anabolic activity. In fact, this invention anticipates, and includes as a prophetic example, those anabolic agents that may be completely devoid of androgenic activity. Examples of such growth hormone-releasing agents include: somatoliberins; growth hormone-releasing hormone active fragments, such as, for example, hGRF (1-29) amide and hexarelin (GHRP-6). Hexarelin is a growth hormone releasing peptide mimetic agent, i.e., it mimics the effects of growth hormone releasing peptide in the body and contains between 2 and 20 amino acids. In particularly preferred embodiments, more than one growth hormone-releasing agent may be used in combination. A preferred combination comprises growth hormone-releasing factor (GRF or GHRH) and a growth hormone releasing peptide or peptidomimetic (GHRP). This combination has been reported to act by separate mechanisms for the release of endogenous growth hormone, and the effects have been shown in some cases to be additive, or even, synergistic, working at a separate receptor often called the Ghrelin receptor, to differentiate it from the GHRH receptor. Since the GHrelin receptor has recently been elucidated, prophetically other ligands for this receptor are anticipated to be synthesized and/or discovered in the future, and these are included by example (Baldelli, R et. al. Endocrine 14 (1):95-99, 2001). These are often referred to as GHSs (growth hormone secretagogue).

The administration of a GH or IGF-1 secretagogue will reduce plasma androgen concentration in humans (Tapanainem J et.al, Fertility and Sterility 58: 726-732). This effect increases the need for exogenous androgen, such as testosterone, to be also administered as a co-treatment to restore and amplify existing levels.

Other compounds are known to affect this system which is known as the hypothalamo-pituitary-hepatic axis for GH, among other terms. Prophetically, it is probable that other compounds involved in this hormonal regulatory system may play a role in indirectly or directly influencing and increasing levels of GH, IGF-1, or IGF-2, and may be administered in the context of this invention along with the androgenic supplementation to get maximal effects of the growth/anti-aging effects of such treatment. Other indications that may be treated besides fibromyalgia may be syndromes affecting the growth of individuals, including but not limited to pituitary dwarfism, conditions or syndromes that are well known to practitioners in the field of endocrinology, growth, and aging.

For the administration of the GH agents that are described in detail above, they may be administered by a variety of means. These agents may be administered separately from the androgen administration, using the modalities of intranasal, transdermal, parenteral (subcutaneous or intravenous), or oral (with or without permeation enhancement and preferably with enteric protection, since proteins and peptides may be degraded by gastric exposure). GH itself is most preferably administered by parenteral means in practice, because it is a large protein that is of limited stability and limited absorption. However, intranasal administration is also an acceptable means for this and other large proteins or peptides. After the administration modality is chosen for the GH agent, the androgen may be administered in a separate treatment with a different regimen. The desired method for androgen administration is preferably oral, transdermal, intravaginal, or intranasal delivery, although it is most preferred to be administered transdermally in the form of a gel or patch. The literature is replete with examples of compositions suitable in the context of this disclosure for the transdermal administration of these compounds in solution, gel, emulsion, or patch forms.

In addition to a separate delivery modality for the GH agent and the androgenic compound selected for treatment, the two may be combined in a single combination therapy. For example, both could be incorporated together in an oral form, tablet, or suspension, with the caveat that any proteinaceous agent is suitably protected from gastric degradation. Alternatively, the combination of agents may be administered intranasally in one unit through separate delivery chambers, known to those of skill in intranasal delivery, or together in the same liquid, semi-solid, or solid delivery form. For example, a microparticulate or nanoparticulate dry solid system could be administered intranasally. Or the combined agents could be both administered transdermally. The two treatments could be incorporated together in a patch, or most preferably in a topical liquid or semi-solid (gel) delivery system. This latter method is most effectively realized in practice for GH agents of the secretagogue (GHSs) variety, such as GHRPs or GHRHs or suitable GHRH fragments that still retain the necessary GH releasing activity. The reason for the suitability is based on the molecular size. It is known throughout the literature that smaller molecules have a higher potential for transdermal delivery than large molecules, such as oligopeptides including GH and IGF-1. The GHrelins and GHRH secretagogues are most preferably selected for the transdermal route based upon small molecular size, such as hexarelin, since transdermal delivery efficiency is good for a hexapeptide. In general, it is preferred that peptides below 30 amino acids are considered for the transdermal delivery format.

Additional clinical studies to confirm the ability of androgen therapy combined with these other hormones to alleviate the symptoms of FMS will be performed. In these studies, the ability of the combined therapy to resolve muscle pain in peri/postmenopausal women diagnosed with FMS will be evaluated. More specifically, patients will be examined for an inverse correlation between serum hormone levels and diminishment in muscle pain. The study will be designed to be similar to the study discussed above in this application. Patients will be assigned randomly to one of the following regimens: 1) placebo twice a day for two months; 2) combination testosterone therapy comprising testosterone and the hormone for testing (e.g., growth hormone) for two months; 3) testosterone for 2 months; or 4) test hormone for two months. These treatments will be followed by a one month washout phase and the patients will again be randomly assigned to one of the above treatment regimens for another two month period.

Patients will be provided with a Patient Questionnaire Form to fill out to assess their symptoms and level of pain in a semi-quantitative manner at the baseline, 2 month and 5 month timepoints. Included in the questionnaire are parameters for patients to evaluate that are common to published and validated FMS patient questionnaires such as sleeplessness, fatigue, headache and stiffness (Wolfe et al., Arthritis and Rheumatism, 1990, 33(2):160-172; Goldenberg et al., Arthritis and Rheumatism, 1996, 39(11):1852-9; and Burckhardt et al., J. Rheumatology, 1991, 18:728-33). The attending physician will also complete a Physician's Form at the baseline, 2 month and 5 month time points to verify that the patient fulfills the criteria for FMS by the American College of Rheumatology, and to document the intensity of the muscle pain for each of the 18 commonly recognized tender points that patients with FMS are known to have.

Patients will be tested at the baseline, 2 month and 5 month time points for total serum hormone levels, serum estradiol levels, cardiac health and liver function. Patients will be tested at a common time of day, preferably a predetermined peak time for the androgen, after fasting since midnight, and on day 3 after the start of their menstrual period if they are still menstruating.

Clinical studies will also be performed to confirm the ability of androgen therapy combined with these other hormones to alleviate the symptoms of muscle pain and fatigue in patients with muscle atrophy in older patients, wherein the muscle pain and fatigue can be attributed to muscle atrophy. In these studies, the ability of the combined therapy to resolve muscle pain in older individuals experiencing symptoms of muscle pain and/or fatigue due to muscle atrophy will be evaluated. More specifically, patients will be examined for an inverse correlation between serum hormone levels and diminishment in muscle pain. The study will be designed to be similar to the study discussed above in this application. The population for study initially will be patients of either sex over 50 years of age who have been shown to have muscle atrophy due to either aging, disease associated with muscle atrophy, as in disease- or stress-associated wasting syndrome (e.g., cancer or any other chronic disease state), or muscle atrophy due to prolonged muscle inactivity (e.g., accident). Patients will be assigned randomly to one of the following regimens: 1) placebo twice a day for two months; 2) combination testosterone therapy comprising testosterone and the hormone for testing (e.g., growth hormone)for two months; 3) testosterone for 2 months; or 4) test hormone for two months. These treatments will be followed by a one month washout phase and the patients will again be randomly assigned to one of the above treatment regimens for another two month period. Patients will be provided with a Patient Questionnaire Form to fill out to assess their symptoms and level of pain in a semi-quantitative manner at the baseline, 2 month and 5 month timepoints. Included in the questionnaire are parameters for patients to evaluate are symptoms such as sleeplessness, fatigue, headache and stiffness. The attending physician will also complete a Physician's Form at the baseline, 2 month and 5 month time points to document the intensity of the muscle pain for commonly recognized tender points.

Patients will be tested at the baseline, 2 month and 5 month time points for total serum hormone levels, serum estradiol levels, cardiac health and liver function. Patients will be tested at a common time of day, preferably a predetermined peak time for the androgen, after fasting since midnight, and between d3-12 (proliferative or follicular phase of the menstrual cycle) after the start of their menstrual period if they are still menstruating.

What is claimed is:

1. A method of alleviating the symptoms of muscle pain or muscle wasting in general comprising administering to a patient suffering from muscle pain or muscle wasting an effective amount of an androgen so that the symptoms are alleviated.

2. The method of claim 1 wherein the androgen comprises a testosterone derivative or a growth hormone derivative.

3. The method of claim 1 wherein a combination of androgens is administered to the patient.

4. The method of claim 3 wherein the combination of androgens comprises testosterone or a testosterone derivative and dehydroepiandrosterone.

5. The method of claim 1 wherein a combination of an androgen and a growth hormone is administered to the patient.

6. The method of claim 1 wherein a combination of an androgen and a compound that increases levels of growth hormone in blood is administered to the patient.

7. The method of claim 6 wherein the compound is selected from the group consisting of growth hormone releasing peptides, growth hormone releasing hormone, IGF-1, and IGF-2.

8. The method of claim 6 wherein the compound is a growth hormone releasing peptide mimetic compound that is between 2 and 20 amino acids in length and is known to release growth hormone.

9. The method of claim 8 wherein the growth hormone releasing peptide mimetic is hexarelin or equivalent peptide.
